(19) United States
(12) Patent Application Publication
(10) Pub. No.: US 2022/0044061 A1
(43) Pub. Date: Feb. 10, 2022

(54) DATA LABELING MODEL TRAINING METHOD, ELECTRONIC DEVICE AND STORAGE MEDIUM

(71) Applicant: HON HAI PRECISION INDUSTRY CO., LTD., New Taipei (TW)

(72) Inventors: Tung-Tso TSAI, New Taipei (TW); Chin-Pin KUO, New Taipei (TW); Wan-Jhen LEE, New Taipei (TW); Guo-Chin SUN, New Taipei (TW)

(21) Appl. No.: 17/393,535

(22) Filed: Aug. 4, 2021

(30) Foreign Application Priority Data
Aug. 6, 2020 (CN) 202010785727.0

(51) Int. Cl. G06K 9/62; G06F 16/535; G06T 7/00

(52) U.S. Cl. CPC G06K 9/628; G06K 9/6256; G06T 7/0012; G06F 16/535; G06K 9/6265

(57) ABSTRACT

A data labeling model training method, an electronic device employing the method, and a storage medium are provided. The method acquires medical image data. An improved quality of the medical image data to be used for training the data labeling model is obtained by filtering the medical data, so as to enable training with higher-quality training material. The data labeling model is used to label medical data with improved efficiency and accuracy.

### FIELD

The present disclosure relates to a technical field of data processing, specifically a data labeling model training method, an electronic device, and a storage medium.

### BACKGROUND

The proper and effective labeling of medical data is necessary, but in practice, it is found that the labeling of medical data requires participation of professionals with professional knowledge, otherwise efficiency is not high.

Therefore, how to improve the efficiency of data labeling is a technical problem that needs to be solved urgently.

### SUMMARY

A data labeling model training method and an electronic device employing the method are provided, which can greatly improve an efficiency of data labeling.

A first aspect of the present disclosure provides a data labeling model training method, the method includes: acquiring medical image data; filtering the medical image data to obtain filtered data; classifying the filtered data to obtain data classified into different categories; acquiring labeling information corresponding to the classified data: forming labeling data according to the category of the classified data, the classified data, and the labeling information; training the labeling data and obtaining a data labeling model.

In some embodiments, after training the labeling data and obtaining a data labeling model, the method further includes: acquiring test data; testing the data labeling model by using the test data and obtaining a test result; when the test result is that the data labeling model is normal, ending the training of the data labeling model.

In some embodiments, the method further includes: when the test result is that the data labeling model is abnormal, determining that the training of the data labeling model is still unfinished; continuing the training of the unfinished data labeling model.

In some embodiments, the method of testing the data labeling model by using the test data and obtaining a test result includes: inputting the test data into the data labeling model and obtaining a first labeling result; determining an accuracy rate of the first labeling result; determining the test result is that the data labeling model is normal, when the accuracy rate is greater than a predetermined accuracy rate threshold; determining the test result is that the data labeling model is abnormal, when the accuracy rate is less than or equal to the predetermined accuracy rate threshold.

In some embodiments, the method further includes: acquiring data to be labeled; using the data labeling model to label the data to be labeled, and obtaining a second labeling result corresponding to the data to be labeled; outputting the second labeling result corresponding to the data to be labeled.

A second aspect of the present disclosure provides an electronic device, the electronic device includes a storage medium and a processor, the storage medium stores at least one computer-readable instruction, and the processor executes the at least one computer-readable instruction to implement to: acquire medical image data; filter the medical image data to obtain filtered data; classify the filtered data to obtain data classified into different categories; acquire labeling information corresponding to the classified data; form labeling data according to the category of the classified data, the classified data, and the labeling information; train the labeling data and obtain a data labeling model.

A third aspect of the present disclosure provides a non-transitory storage medium having stored thereon at least one computer-readable instruction that, when the at least one computer-readable instruction are executed by a processor, implements a data labeling model training method, the method includes: acquiring medical image data; filtering the medical image data to obtain filtered data; classifying the filtered data to obtain data classified into different categories; acquiring labeling information corresponding to the classified data; forming labeling data according to the category of the classified data, the classified data, and the labeling information; training the labeling data and obtaining a data labeling model.

The data labeling model training method, the electronic device, and the storage medium of the present disclosure can improve the quality of the medical image data by filtering the medical image data, thereby training a better data labeling model based on the filtered data. The data labeling model is used to label data with much-improved data labeling efficiency.

### BRIEF DESCRIPTION OF THE DRAWINGS

FIG. 1 shows a flowchart of a data labeling model training method provided in an embodiment of the present disclosure.

FIG. 2 shows a schematic structural diagram of a data labeling model training device provided in an embodiment of the present disclosure.

FIG. 3 shows a schematic structural diagram of an electronic device provided in an embodiment of the present disclosure.

### DETAILED DESCRIPTION

For clarity of the illustration of objectives, features, and advantages of the present disclosure, the drawings combined with the detailed description illustrate the embodiments of the present disclosure hereinafter. It is noted that embodiments of the present disclosure and features of the embodiments can be combined, when there is no conflict.

Various details are described in the following descriptions for a better understanding of the present disclosure, however, the present disclosure may also be implemented in other ways other than those described herein. The scope of the present disclosure is not to be limited by the specific embodiments disclosed below.

Unless defined otherwise, all technical and scientific terms used herein have the same meaning as commonly understood by one of ordinary skill in the art to which the present disclosure belongs. The terms used herein in the present disclosure are only for the purpose of describing specific embodiments and are not intended to limit the present disclosure.

The data labeling model training method of the present disclosure can be applied to several electronic devices. Such electronic devices include hardware such as, but not limited to, a microprocessor and an Application Specific Integrated Circuit (ASIC), Field-Programmable Gate Array (FPGA), Digital Signal Processor (DSP), embedded devices, etc.

Such electronic device may be a device such as a desktop computer, a notebook, a palmtop computer, or a cloud server. The electronic device can interact with users through a keyboard, a mouse, a remote control, a touch panel, or a voice control device.

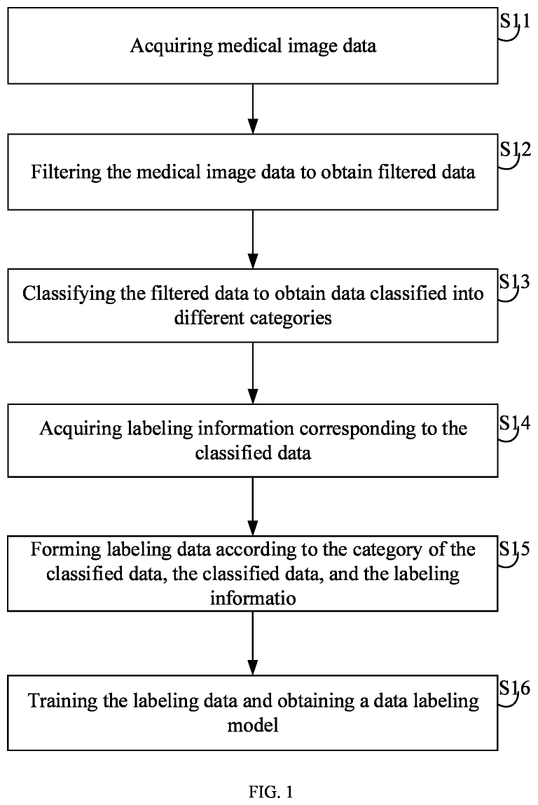

FIG. 1 is a flowchart of a data labeling model training method in an embodiment of the present disclosure. The data labeling model training method is applied to electronic devices. According to different needs, the order of the steps in the flowchart can be changed, and some can be omitted.

In block S11, acquiring medical image data.

In an embodiment of the present disclosure, the medical image data may be textual image data, such as various index values of blood test reports, or data in form of images, such as images of cells.

In block S12, filtering the medical image data to obtain filtered data.

In an embodiment of the present disclosure, the medical image data can be filtered, and medical image data that is not suitable for labeling can be filtered out. Labels can be applied to the remaining medical image data, that is, the filtered data, can be used for labeling with high quality. The data is used to train a model, which can improve an accuracy and a training speed of the model.

In block S13, classifying the filtered data to obtain data classified into different categories.

In an embodiment of the present disclosure, because different types of data need to be labeled differently, the filtered data needs to be classified, so that an efficiency of labeling can be improved. For example, when the medical image data are the images of cells, it is necessary to form a frame around abnormal cells and label them as cancerous cells or otherwise. When the image data are the various index values of blood test reports, the labeling must include classification into different types or qualities of blood.

In block S14, acquiring labeling information corresponding to the classified data.

In an embodiment of the present disclosure, the labeling information can include qualified, unqualified, diseased cells, cancer cells, and borders at designated locations.

In block S15, forming labeling data according to the category of the classified data, the classified data, and the labeling information.

In an embodiment of the present disclosure, for the textual image data, the labeling information can be displayed at a preset position of the textual image data. For the data as to the images of cells, in addition to displaying the labeling information at a preset position, it is also necessary to form a frame on the cell image data according to the position information carried by the labeling information.

In block S16, training the labeling data and obtaining a data labeling model.

In an embodiment of the present disclosure, the data labeling model can be obtained through deep learning training.

In an embodiment, the data labeling model training method further includes:

acquiring test data;

testing the data labeling model by using the test data and obtaining a test result;

when the test result is that the data labeling model is normal, ending the training of the data labeling model.

In the above embodiment, the test data can be used to test the data labeling model to obtain a test result, and the test result is used to indicate whether the data labeling model can be used normally.

In an embodiment, the method further includes:

when the test result is that the data labeling model is abnormal, determining that the training of the data labeling model is still unfinished;

continuing the training of the unfinished data labeling model.

In the above embodiment, when the test result is that the data labeling model is abnormal, it means that the data labeling model cannot be used normally and the training of the data labeling model needs to be continued.

In an embodiment, the method of testing the data labeling model by using the test data and obtaining a test result includes:

inputting the test data into the data labeling model and obtaining a first labeling result;

determining an accuracy rate of the first labeling result;

determining the test result is that the data labeling model is normal, when the accuracy rate is greater than a predetermined accuracy rate threshold;

determining the test result is that the data labeling model is abnormal, when the accuracy rate is less than or equal to the predetermined accuracy rate threshold.

In the above embodiment, the test data can be input into the data labeling model, the first labeling results output by the data labeling model for the test data can be obtained, and then the accuracy rate of the first labeling results can be calculated. A predetermined accuracy rate threshold can be set in advance. When the accuracy rate of the first labeling result is greater than the predetermined accuracy rate threshold (for example, greater than 80%), it is determined that the data labeling model can be used normally. When the accuracy rate of the first labeling result is less than or equal to the predetermined accuracy rate threshold, it is determined that the data labeling model cannot be used normally, that is, it is determined that the data labeling model is abnormal and unfinished.

In an embodiment, the method further includes:

acquiring data to be labeled;

using the data labeling model to label the data to be labeled, and obtaining a second labeling result corresponding to the data to be labeled;

outputting the second labeling result corresponding to the data to be labeled.

In the above embodiment, the data to be labeled can be input into the trained data labeling model to obtain the labeling result corresponding to the data to be labeled, which improves an efficiency of data labeling.

In the flow of method described in FIG. 1, the overall quality of the medical image data used for training the data labeling model can be improved by filtering the medical image data, so as to train a better data labeling model. The data labeling model can be used to label the data, which can improve the efficiency of data labeling.

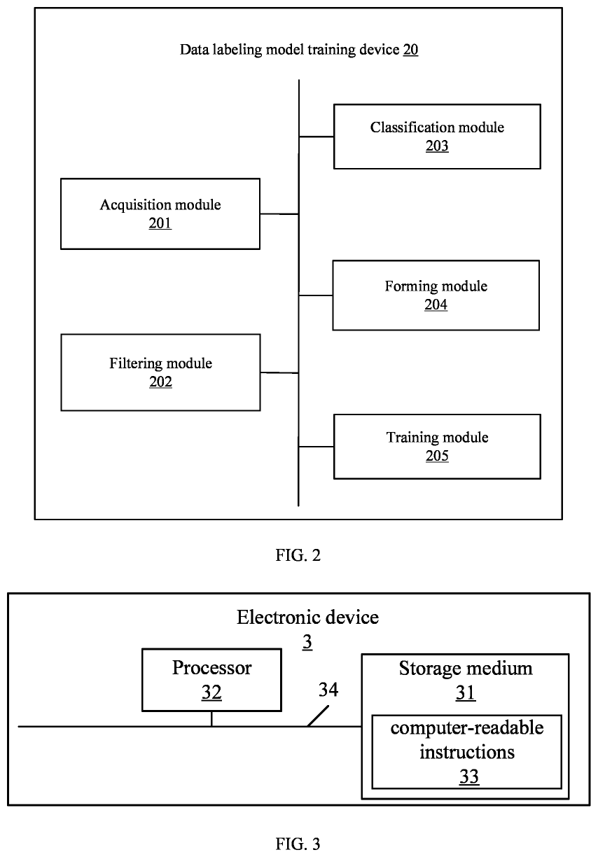

FIG. 2 shows a data labeling model training device provided in the embodiment of the present disclosure.

In some embodiments, the data labeling model training device 20 runs in an electronic device. The data labeling model training device 20 can include a plurality of function modules consisting of program code segments. The program code of each program code segments in the data labeling model training device 20 can be stored in a storage medium and executed by at least one processor to perform data labeling model training.

As shown in FIG. 2, the data labeling model training device 20 can include: an acquisition module 201, a filtering module 202, a classification module 203, a forming module 204, and a training module 205. Modules as referred to in the present disclosure refers to a series of computer-readable instruction segments that can be executed by at least one processor and that are capable of performing fixed functions, which are stored in a storage medium. In some embodiment, the functions of each module will be detailed in the following embodiments.

The above-mentioned integrated unit implemented in functional modules of software can be stored in a non-transitory readable storage medium. The above modules are stored in a storage medium and includes several instructions for causing an electronic device (which can be a personal computer, a dual-screen device, or a network device) or a processor to execute the method described in various embodiments in the present disclosure.

The acquisition module 201 acquires medical image data.

In an embodiment of the present disclosure, the medical image data may be textual image data, such as various index values of blood test reports, or data in form of images, such as images of cells.

The filtering module 202 filters the medical image data to obtain filtered data.

In an embodiment of the present disclosure, the medical image data can be filtered, and medical image data that is not suitable for labeling can be filtered out. The remaining medical image data, that is, the filtered data, can be used for labeling with high quality. The data is used to train a model, which can improve an accuracy and a training speed of the model.

The classification module 203 classifies the filtered data to obtain data classified into different categories.

In an embodiment of the present disclosure, because different types of data need to be labeled differently, the filtered data needs to be classified, so that an efficiency of labeling can be improved. For example, when the medical image data are the images of cells, it is necessary to form a frame around abnormal cells and label them as cancerous cells or otherwise. When the image data are the various index values of blood test reports, the labeling must include classification into different types or qualities of blood.

The acquisition module 201 acquires labeling information corresponding to the classified data.

In an embodiment of the present disclosure, the labeling information can include qualified, unqualified, diseased cells, cancer cells, and borders at designated locations.

The forming module 204 forms labeling data according to the category of the classified data, the classified data, and the labeling information.

In an embodiment of the present disclosure, for the textual image data, the labeling information can be displayed at a preset position of the textual image data. For the data as to the images of cells, in addition to displaying the labeling information at a preset position, it is also necessary to form a frame on the cell image data according to the positional information carried by the labeling information.

The training module 205 trains the labeling data and obtains a data labeling model.

In an embodiment of the present disclosure, the data labeling model can be obtained through deep learning training.

In an embodiment, the acquisition module 201 configured to acquire test data, after the training module 205 trains the labeling data and obtains a data labeling model.

The data labeling model training device 20 further includes a testing module and a determination module. The testing module tests the data labeling model by using the test data and obtaining a test result.

The determination module configured to, when the test result is that the data labeling model is normal, end the training of the data labeling model.

In the above embodiment, the test data can be used to test the data labeling model to obtain a test result, and the test result is used to indicate whether the data labeling model can be used normally.

In an embodiment, the determination module further configured to, when the test result is that the data labeling model is abnormal, determining that the training of the data labeling model is still unfinished.

The training module 205 continues the training of the unfinished data labeling model.

In the above embodiment, when the test result is that the data labeling model is abnormal, it means that the data labeling model cannot be used normally and the training of the data labeling model needs to be continued.

In an embodiment, the testing module testing the data labeling model by using the test data and obtaining a test result includes:

inputting the test data into the data labeling model and obtaining a first labeling result;

determining an accuracy rate of the first labeling result;

determining the test result is that the data labeling model is normal, when the accuracy rate is greater than a predetermined accuracy rate threshold;

determining the test result is that the data labeling model is abnormal, when the accuracy rate is less than or equal to the predetermined accuracy rate threshold.

In the above embodiment, the test data can be input into the data labeling model, the first labeling results output by the data labeling model for the test data can be obtained, and then the accuracy rate of the first labeling results can be calculated. A predetermined accuracy rate threshold can be set in advance. When the accuracy rate of the first labeling result is greater than the predetermined accuracy rate threshold (for example, greater than 80%), it is determined that the data labeling model can be used normally. When the accuracy rate of the first labeling result is less than or equal to the predetermined accuracy rate threshold, it is determined that the data labeling model cannot be used normally, that is, it is determined that the data labeling model is abnormal and unfinished.

In an embodiment, the acquisition module 201 further configured to acquire data to be labeled, after the determination module ends the training of the data labeling model.

The data labeling model training device 20 further includes a labeling module and an output module. The labeling module uses the data labeling model to label the data to be labeled, and obtains a second labeling result corresponding to the data to be labeled;

The output module outputs the second labeling result corresponding to the data to be labeled.

In the above embodiment, the data to be labeled can be input into the trained data labeling model to obtain the labeling result corresponding to the data to be labeled, which improves an efficiency of data labeling.

In the data labeling model training device 20 described in FIG. 2, the overall quality of the medical image data used for training the data labeling model can be improved by filtering the medical image data, so as to train a better data labeling model. The data labeling model can be used to label the data, which can improve the efficiency of data labeling.

The embodiment provides a non-transitory readable storage medium having computer-readable instructions stored therein. The computer-readable instructions are executed by a processor to implement the steps in the above-mentioned data labeling model training method, such as in steps in block S10-S16 shown in FIG. 1:

In block S11, acquiring medical image data;

In block S12, filtering the medical image data to obtain filtered data;

In block S13, classifying the filtered data to obtain data classified into different categories;

In block S14, acquiring labeling information corresponding to the classified data;

In block S15, forming labeling data according to the category of the classified data, the classified data, and the labeling information;

In block S16, training the labeling data and obtaining a data labeling model.

Or, the computer-readable instruction being executed by the processor to realize the functions of each module/unit in the above-mentioned device embodiments, such as the modules 201-205 in FIG. 2:

The acquisition module 201 acquires medical image data;

The filtering module 202 filters the medical image data to obtain filtered data;

The classification module 203 classifies the filtered data to obtain data classified into different categories;

The forming module 204 forms labeling data according to the category of the classified data, the classified data, and the labeling information;

The training module 205 trains the labeling data and obtains a data labeling model.

FIG. 3 is a schematic structural diagram of an electronic device provided in embodiment four of the present disclosure. The electronic device 3 may include: a storage medium 31, at least one processor 32, computer-readable instructions 33 stored in the storage medium 31 and executable on the at least one processor 32, for example, data labeling model training programs, and at least one communication bus 34. The processor 32 executes the computer-readable instructions to implement the steps in the embodiment of the data labeling model training method, such as in steps in block S11-S16 shown in FIG. 1. Alternatively, the processor 32 executes the computer-readable instructions to implement the functions of the modules/units in the foregoing device embodiments, such as the modules 201-205 in FIG. 2.

Exemplarily, the computer-readable instructions can be divided into one or more modules/units, and the one or more modules/units are stored in the storage medium 31 and executed by the at least one processor 32. The one or more modules/units can be a series of computer-readable instruction segments capable of performing specific functions, and the instruction segments are used to describe execution processes of the computer-readable instructions in the electronic device 3. For example, the computer-readable instruction can be divided into the acquisition module 201, the filtering module 202, the classification module 203, the forming module 204, and the training module 205, as in FIG. 2.

The electronic device 3 can be a device such as a desktop computer, a notebook, a palmtop computer, and a cloud server. Those skilled in the art will understand that the schematic diagram 3 is only an example of the electronic device 3 and does not constitute a limitation on the electronic device 3. Another electronic device 3 may include more or have fewer components than shown in the figures or may combine some components or have different components. For example, the electronic device 3 may further include an input/output device, a network access device, a bus, and the like.

The at least one processor 32 can be a central processing unit (CPU), or can be another general-purpose processor, digital signal processor (DSPs), application-specific integrated circuit (ASIC), Field-Programmable Gate Array (FPGA), another programmable logic device, discrete gate, transistor logic device, or discrete hardware component, etc. The processor 32 can be a microprocessor or any conventional processor. The processor 32 is a control center of the electronic device 3 and connects various parts of the entire electronic device 3 by using various interfaces and lines.

The storage medium 31 can be configured to store the computer-readable instructions and/or modules/units. The processor 32 may run or execute the computer-readable instructions and/or modules/units stored in the storage medium 31 and may call up data stored in the storage medium 31 to implement various functions of the electronic device 3. The storage medium 31 mainly includes a storage program area and a storage data area. The storage program area may store an operating system, and an application program required for at least one function (such as a sound playback function, an image playback function, etc.), etc. The storage data area may store data (such as audio data, a phone book, etc.) created according to the use of the electronic device 3. In addition, the storage medium 31 may include a high-speed random access storage medium, and may also include a non-transitory storage medium, such as a hard disk, an internal storage medium, a plug-in hard disk, a smart media card (SMC), a secure digital (SD) Card, a flashcard, at least one disk storage device, a flash storage medium device, or another non-transitory solid-state storage device.

When the modules/units integrated into the electronic device 3 are implemented in the form of software functional units having been sold or used as independent products, they can be stored in a non-transitory readable storage medium. Based on this understanding, all or part of the processes in the methods of the above embodiments implemented by the present disclosure can also be completed by related hardware instructed by computer-readable instructions. The computer-readable instructions can be stored in a non-transitory readable storage medium. The computer-readable instructions, when executed by the processor, may implement the steps of the foregoing method embodiments. The computer-readable instructions include computer-readable instruction codes, and the computer-readable instruction codes can be in a source code form, an object code form, an executable file, or some intermediate form. The non-transitory readable storage medium can include any entity or device capable of carrying the computer-readable instruction code, such as a recording medium, a U disk, a mobile hard disk, a magnetic disk, an optical disk, a computer storage medium, or a read-only storage medium (ROM).

In the several embodiments provided in the preset application, it should be understood that the disclosed electronic device and method can be implemented in other ways. For example, the embodiments of the devices described above are merely illustrative. For example, divisions of the units are only logical function divisions, and there can be other manners of division in actual implementation.

In addition, each functional unit in each embodiment of the present disclosure can be integrated into one processing unit, or can be physically present separately in each unit or two or more units can be integrated into one unit. The above modules can be implemented in a form of hardware or in a form of a software functional unit.

The present disclosure is not limited to the details of the above-described exemplary embodiments, and the present disclosure can be embodied in other specific forms without departing from the spirit or essential characteristics of the present disclosure. Therefore, the present embodiments are to be considered as illustrative and not restrictive, and the scope of the present disclosure is defined by the appended claims. All changes and variations in the meaning and scope of equivalent elements are included in the present disclosure. Any reference sign in the claims should not be construed as limiting the claim. Furthermore, the word “comprising” does not exclude other units nor does the singular exclude the plural. A plurality of units or devices stated in the system claims may also be implemented by one unit or device through software or hardware. Words such as “first” and “second” are used to indicate names, but not in any particular order.

Finally, the above embodiments are only used to illustrate technical solutions of the present disclosure and are not to be taken as restrictions on the technical solutions. Although the present disclosure has been described in detail with reference to the above embodiments, those skilled in the art should understand that the technical solutions described in one embodiment can be modified, or some of the technical features can be equivalently substituted, and that these modifications or substitutions are not to detract from the essence of the technical solutions or from the scope of the technical solutions of the embodiments of the present disclosure.

What is claimed is:

1. A data labeling model training method, the method comprising:
acquiring medical image data;
filtering the medical image data to obtain filtered data;
classifying the filtered data to obtain data classified into different categories;
acquiring labeling information corresponding to the classified data;
forming labeling data according to the category of the classified data, the classified data, and the labeling information;
training the labeling data and obtaining a data labeling model.

2. The data labeling model training method according to claim 1, after training the labeling data and obtaining a data labeling model, the method further comprising:
acquiring test data;
testing the data labeling model by using the test data and obtaining a test result;
when the test result is that the data labeling model is normal, ending the training of the data labeling model.

3. The data labeling model training method according to claim 2, the method further comprising:
when the test result is that the data labeling model is abnormal, determining that the training of the data labeling model is still unfinished;
continuing the training of the unfinished data labeling model.

4. The data labeling model training method according to claim 2, wherein testing the data labeling model by using the test data and obtaining a test result comprises:
inputting the test data into the data labeling model and obtaining a first labeling result;
determining an accuracy rate of the first labeling result;
determining the test result is that the data labeling model is normal, when the accuracy rate is greater than a predetermined accuracy rate threshold;
determining the test result is that the data labeling model is abnormal, when the accuracy rate is less than or equal to the predetermined accuracy rate threshold.

5. The data labeling model training method according to claim 1, the method further comprising:
acquiring data to be labeled;
using the data labeling model to label the data to be labeled, and obtaining a second labeling result corresponding to the data to be labeled;
outputting the second labeling result corresponding to the data to be labeled.

6. The data labeling model training method according to claim 2, the method further comprising:
acquiring data to be labeled;
using the data labeling model to label the data to be labeled, and obtaining a second labeling result corresponding to the data to be labeled;
outputting the second labeling result corresponding to the data to be labeled.

7. The data labeling model training method according to claim 3, the method further comprising:
acquiring data to be labeled;
using the data labeling model to label the data to be labeled, and obtaining a second labeling result corresponding to the data to be labeled;
outputting the second labeling result corresponding to the data to be labeled.

8. An electronic device comprising a storage medium and a processor, the storage medium stores at least one computer-readable instruction, and the processor executes the at least one computer-readable instruction to implement to:
acquire medical image data;
filter the medical image data to obtain filtered data;
classify the filtered data to obtain data classified into different categories;
acquire labeling information corresponding to the classified data;
form labeling data according to the category of the classified data, the classified data, and the labeling information;
train the labeling data and obtaining a data labeling model.

9. The electronic device according to claim 8, wherein the processor converting a data type of the initial model by:
acquiring test data;
testing the data labeling model by using the test data and obtaining a test result;
when the test result is that the data labeling model is normal, ending the training of the data labeling model.

10. The electronic device according to claim 9, wherein the processor is further to:
when the test result is that the data labeling model is abnormal, determine that the training of the data labeling model is still unfinished;
continue the training of the unfinished data labeling model.

11. The electronic device according to claim 9, wherein the processor testing the data labeling model by using the test data and obtaining a test result by:
inputting the test data into the data labeling model and obtaining a first labeling result;
determining an accuracy rate of the first labeling result;
determining the test result is that the data labeling model is normal, when the accuracy rate is greater than a predetermined accuracy rate threshold;
determining the test result is that the data labeling model is abnormal, when the accuracy rate is less than or equal to the predetermined accuracy rate threshold.

12. The electronic device according to claim 8, wherein the processor is further to:
acquire data to be labeled;
use the data labeling model to label the data to be labeled, and obtain a second labeling result corresponding to the data to be labeled;
output the second labeling result corresponding to the data to be labeled.

13. The electronic device according to claim 9, wherein the processor is further to:
acquire data to be labeled;
use the data labeling model to label the data to be labeled, and obtain a second labeling result corresponding to the data to be labeled;
output the second labeling result corresponding to the data to be labeled.

14. The electronic device according to claim 10, wherein the processor is further to:
acquire data to be labeled;
use the data labeling model to label the data to be labeled, and obtain a second labeling result corresponding to the data to be labeled;
output the second labeling result corresponding to the data to be labeled.

15. A non-transitory storage medium having stored thereon at least one computer-readable instruction that, when the at least one computer-readable instruction are executed by a processor to implement the following steps:
acquiring medical image data;
filtering the medical image data to obtain filtered data;
classifying the filtered data to obtain data classified into different categories;
acquiring labeling information corresponding to the classified data;
forming labeling data according to the category of the classified data, the classified data, and the labeling information;
training the labeling data and obtaining a data labeling model.

16. The non-transitory storage medium according to claim 15, after training the labeling data and obtaining a data labeling model, the method further comprising:
acquiring test data;
testing the data labeling model by using the test data and obtaining a test result;
when the test result is that the data labeling model is normal, ending the training of the data labeling model.

17. The non-transitory storage medium according to claim 16, the method further comprising:
when the test result is that the data labeling model is abnormal, determining that the training of the data labeling model is still unfinished;
continuing the training of the unfinished data labeling model.

18. The non-transitory storage medium according to claim 16, wherein testing the data labeling model by using the test data and obtaining a test result comprises:
inputting the test data into the data labeling model and obtaining a first labeling result;
determining an accuracy rate of the first labeling result;
determining the test result is that the data labeling model is normal, when the accuracy rate is greater than a predetermined accuracy rate threshold;
determining the test result is that the data labeling model is abnormal, when the accuracy rate is less than or equal to the predetermined accuracy rate threshold.

19. The non-transitory storage medium according to claim 15, the method further comprising:
acquiring data to be labeled;
using the data labeling model to label the data to be labeled, and obtaining a second labeling result corresponding to the data to be labeled;
outputting the second labeling result corresponding to the data to be labeled.

20. The non-transitory storage medium according to claim 16, the method further comprising:
acquiring data to be labeled;
using the data labeling model to label the data to be labeled, and obtaining a second labeling result corresponding to the data to be labeled;
outputting the second labeling result corresponding to the data to be labeled.
